(19) United States
(12) Patent Application Publication
(10) Pub. No.: US 2024/0099620 A1
(43) Pub. Date: Mar. 28, 2024

(54) METHOD AND APPARATUS FOR CHECKING OF MENTAL HEALTH USING CONTENTS

(71) Applicant: AJOU UNIVERSITY INDUSTRY-ACADEMIC COOPERATION FOUNDATION, Gyeonggi-Do (KR)

(72) Inventors: Tae-Sun Chung, Gyeonggi-Do (KR); Kamil Zeberga Kerbaga, Gyeonggi-Do (KR)

(73) Assignee: AJOU UNIVERSITY INDUSTRY-ACADEMIC COOPERATION FOUNDATION, Gyeonggi-Do (KR)

(21) Appl. No.: 18/244,044

(22) Filed: Sep. 8, 2023

(30) Foreign Application Priority Data
Sep. 28, 2022 (KR) 10-2022-0123372

(51) Int. Cl. A61B 5/16; G06F 40/166; G06F 40/268; G06F 40/284

(52) U.S. Cl. CPC A61B 5/165; G06F 40/166; G06F 40/268; G06F 40/284

(57) ABSTRACT

The present invention relates to a method and apparatus for checking mental health using contents, and may include collecting text data, which is content uploaded to at least one service server providing a social network service, by an electronic apparatus, performing preprocessing by which the electronic apparatus removes obsolete text from the text data and converts the extracted meaningful text to lowercase letters, performing, by the electronic apparatus, labeling of preprocessed meaningful text, performing, by the electronic apparatus, word embedding for the labeled meaningful text, and checking the mental health status of a user who has uploaded the text data by applying the word embedding result to a deep learning algorithm by the electronic apparatus, and it is possible to apply to other exemplary embodiments.

### CROSS-REFERENCE TO RELATED APPLICATION

This application claims priority to and the benefit of Korean Patent Application No. 10-2022-0123372, filed on Sep. 28, 2022, the disclosure of which is incorporated herein by reference in its entirety.

### TECHNICAL FIELD

The present invention relates to a method and apparatus for checking mental health using contents.

### Background Art

Depression is a disease that causes a variety of cognitive and psychosomatic symptoms with low motivation and depressed mood as the main symptoms, resulting in a decrease in daily function. Depression has a lifetime prevalence of 15% and reaches about 25% especially for women, and it is a serious disease that causes changes in emotions, thoughts, physical condition and behavior. Since depression can be improved to a great extent with the attention of acquaintances or the help of experts, early detection is very important. However, many of depression patients are unaware of the onset of depression or experience long-term suffering because they cannot receive help from acquaintances or experts, and in serious cases, they even end up with extreme choices such as suicide. Further, in many cases, depression is caused by not being able to resolve long-term continuous depression rather than a single major shock event.

As a method for diagnosing such depression, most of patients rely on surveys (Beck Depression Inventory, Edinburgh Postnatal Depression Scale, Postpartum Depression Screening Scale, etc.) or expert counseling due to the nature of the disease showing emotional symptoms. However, these questionnaires are difficult to distinguish between temporary discomfort and clinically meaningful diseases, and people who feel depressed must actively participate in questionnaires or counseling, and thus, there have been problems in that they are difficult to apply to those with early signs of depression or those who are in serious depression. Accordingly, mobile mental health care service attempts have recently been expanding in order to reduce medical costs and improve treatment efficiency.

However, since these mobile mental health care services generally use a self-report questionnaire, there is a problem in that diagnosis errors may occur depending on differences in individual perception of depressed emotions in that they measure depression subjectively. In addition, a problem arises in that the time required for the diagnosis process of depression increases.

### DISCLOSURE

### Technical Problem

The exemplary embodiments of the present invention to solve these conventional problems are directed to providing a method and apparatus for checking mental health using contents that can check mental health such as depression by analyzing user contents that are uploaded to social media by using deep learning.

### Technical Solution

The method for checking mental health using contents according to an exemplary embodiment of the present invention may include: collecting text data, which is content uploaded to at least one service server providing a social network service, by an electronic apparatus; performing preprocessing by which the electronic apparatus removes obsolete text from the text data and converts the extracted meaningful text to lowercase letters; performing, by the electronic apparatus, labeling of the preprocessed meaningful text; performing, by the electronic apparatus, word embedding for the labeled meaningful text; and checking the mental health status of a user who has uploaded the text data by applying the word embedding result to a deep learning algorithm by the electronic apparatus.

In addition, the performing preprocessing may include dividing a paragraph into a plurality of sentences when the text data is a paragraph.

In addition, the performing preprocessing may include removing obsolete text including hash tags, special characters, numbers and spaces from the text data; tokenizing by classifying at least one text included in the text data into words; and converting the meaningful text into lowercase letters.

In addition, the tokenizing may include removing meaningless text including pronouns, prepositions, conjunctions, articles and URLs from the text data; checking a headword or morpheme based on at least one text classified as the word; and converting slang and emoticons included in the text data into words having the same meaning.

In addition, the method may further include displaying parts of speech including nouns, adjectives, adverbs, determiners and conjunctions in the meaningful text.

In addition, the performing labelling of the meaningful text may include generating a text corpus based on the meaningful text; labeling the text corpus for each social network service; performing keyword-based labeling based on a circumplex model of emotions; and classifying the text corpus according to emotion based on the labeling.

In addition, the performing the word embedding is a performing the word embedding by applying the labeled meaningful text to a BERT algorithm, which is the deep learning algorithm.

Moreover, the apparatus for checking mental health using contents according to an exemplary embodiment of the present invention may include a communication unit for collecting text data, which is content uploaded to a service server through communication with at least one service server providing social network service; and a control unit for performing preprocessing to convert meaningful text extracted by removing obsolete text from the text data into lowercase letters, labelling the preprocessed meaningful text, and checking the mental health status of a user, who has uploaded the text data, by applying a word embedding result for the labeled meaningful text to a deep learning algorithm.

In addition, when the text data is a paragraph, the control unit may divide the paragraph into a plurality of sentences.

In addition, the control unit may remove obsolete text including hash tags, special characters, numbers and spaces from the text data, and tokenize by classifying at least one text included in the text data into words.

In addition, the control unit may remove meaningless text including pronouns, prepositions, conjunctions, articles and URLs from the text data, check a headword or morpheme based on at least one text classified as the word, and convert slang and emoticons included in the text data into words having the same meaning to perform the tokenizing.

In addition, the control unit may display parts of speech including nouns, adjectives, adverbs, determiners and conjunctions in the meaningful text.

In addition, the control unit may convert the meaningful text into lowercase letters.

In addition, the control unit may generate a text corpus based on the meaningful text, perform labeling of the text corpus for each social network service, perform keyword-based labeling based on a circumplex model of emotions, and classify the text corpus according to emotion based on the labeling.

In addition, the control unit may perform the word embedding by applying the labeled meaningful text to a BERT algorithm, which is the deep learning algorithm.

### Advantageous Effects

As described above, the method and apparatus for checking mental health using contents according to the present invention analyze user contents that are uploaded to social media by using deep learning to check mental health such as depression, thereby minimizing the hassle of having to visit a psychiatrist, and there is an effect of minimizing diagnosis errors through self-report questionnaires.

### DESCRIPTION OF DRAWINGS

FIG. 1 is a diagram showing the system for checking mental health using contents according to an exemplary embodiment of the present invention.

FIG. 2 is a diagram showing the main configuration of a monitoring apparatus for checking mental health using contents according to an exemplary embodiment of the present invention.

FIG. 3 is a flowchart for explaining the method of checking mental health using contents according to an exemplary embodiment of the present invention.

FIG. 4 is a detailed flowchart for explaining the method of preprocessing data according to an exemplary embodiment of the present invention.

FIG. 5 is a detailed flowchart for explaining the method of labeling data according to an exemplary embodiment of the present invention.

FIG. 6 is a diagram for explaining the Bi-LSTM operation according to an exemplary embodiment of the present invention.

FIG. 7 is a diagram for explaining text representation, dimensionality reduction, and depression and anxiety sensing operations according to an exemplary embodiment of the present invention.

FIG. 8 is a diagram showing the detailed structure for applying knowledge distillation in a pre-trained BERT according to an exemplary embodiment of the present invention.

FIG. 9 is a diagram showing the circumplex model of emotions according to an exemplary embodiment of the present invention.

### MODES OF THE INVENTION

Hereinafter, preferred exemplary embodiments according to the present invention will be described in detail with reference to the accompanying drawings. The detailed description set forth below in conjunction with the accompanying drawings is intended to describe the exemplary embodiments of the present invention and is not intended to represent the only exemplary embodiments in which the present invention may be practiced. In order to clearly describe the present invention in the drawings, parts that are irrelevant to the description may be omitted, and the same reference numerals may be used for the same or similar components throughout the specification.

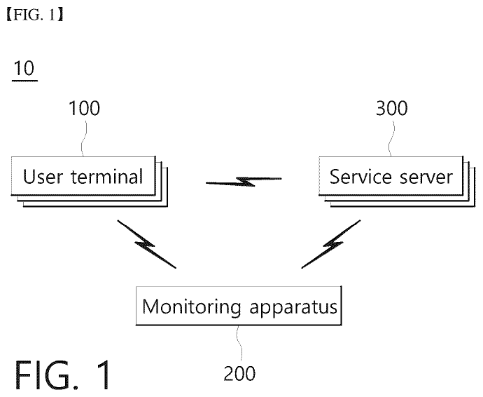

FIG. 1 is a diagram showing the system for checking mental health using contents according to an exemplary embodiment of the present invention.

Referring to FIG. 1, the system 10 according to the present invention may include a plurality of user terminals 100, a monitoring apparatus 200 and a service server 300.

The user terminal 100 is a terminal which is capable of using social network services provided by the service server 300 through communication with the service server 300, and it may be an electronic apparatus such as a smart phone, a tablet PC or the like. The user terminal 100 may access the service server 300 and upload contents such as video data, still image data, text data and the like. In the present invention, text data is used in combination with contents.

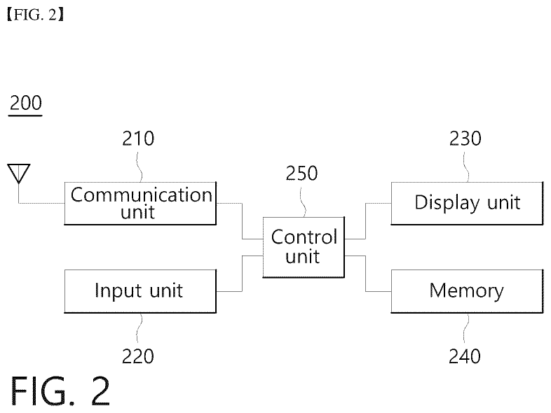

The monitoring apparatus 200 is a apparatus for collecting text data, which is content that is uploaded to at least one service server 300 providing a social network service, analyzing the text data, and checking the mental health status of a user, who has uploaded the text data, by applying the analysis result to a deep learning algorithm, and it may be an electronic apparatus such as a computer or the like. The more detailed operation of the monitoring apparatus 200 will be described with reference to FIG. 2 below.

The service server 300 is a server for providing a social network service, and it may be an electronic apparatus such as a computer or the like. The service server 300 may collect text data uploaded from the user terminal 100 for each user and transmit the collected text data to the monitoring apparatus 200. The service server 300 may include a plurality of servers that respectively provide various social network services such as Instagram, Twitter, TikTok and the like.

FIG. 2 is a diagram showing the main configuration of a monitoring apparatus for checking mental health using contents according to an exemplary embodiment of the present invention.

Referring to FIG. 2, the monitoring apparatus 200 according to the present invention may include a communication unit 210, an input unit 220, a display unit 230, a memory 240 and a control unit 250.

In order to check the mental health of a user through the analysis of content, for example, text data that the user uploads to the service server 300, the communication unit 210 may receive text data that is uploaded to the service server 300 through communication with the service server 300 and provide the text data to the control unit 250. In addition, the communication unit 210 may transmit a confirmation result related to the mental health of the user to the user terminal 100 through communication with the user terminal 100. To this end, the communication unit 210 may perform wireless communication such as 5^{th} generation communication (5G), long term evolution (LTE), long term evolution-advanced (LTE-A), wireless fidelity (Wi-Fi) and the like.

The input unit 220 generates input data in response to a user input of the monitoring apparatus 200. To this end, the input unit 220 may include an input apparatus such as a keyboard, mouse, keypad, dome switch, touch panel, touch keys and buttons.

The display unit 230 outputs output data according to the operation of the monitoring apparatus 200. To this end, the display unit 230 may include a display apparatus such as a liquid crystal display (LCD), a light emitting diode (LED) display, an organic LED (OLED) display or the like. Moreover, the display unit 230 may be combined with the input unit 220 and implemented in the form of a touch screen.

The memory 240 may store various programs for operating the monitoring apparatus 200. In particular, the memory 240 may analyze text data received from the service server 300 through the communication unit 210 to store various algorithms for checking the mental health of a user.

The control unit 250 collects content, that is, text data, from at least one service server 300 that provides different social network services. In this case, the text data may refer to text data that is uploaded to the service server 300 by using at least one user terminal 100.

The control unit 250 performs preprocessing on the collected text data. More specifically, if the collected text data is not a sentence, for example, a paragraph, the control unit 250 divides the collected text data into at least one sentence. The control unit 250 analyzes the text data in units of sentences and deletes obsolete text. For example, the control unit 250 deletes obsolete text such as hash tags, special characters, numbers and spaces that are included in the text data in order to filter and organize messy text data.

The control unit 250 removes meaningless text such as pronouns, prepositions, symbols, conjunctions, articles and URLs from text data. The control unit 250 may classify the text data from which meaningless text has been removed to check headwords or morphemes, and convert slang and emoticons that are included in the text data into words with the same meaning to perform tokenization.

The control unit 250 converts all words, for example, words identified as headwords or morphemes, slang words and words converted from emoticons to lowercase letters to have a certain format in order to avoid confusion that may occur during text data analysis. Moreover, the control unit 250 may tag parts of speech such as nouns, verbs, adjectives, adverbs, determiners and conjunctions to the words that are converted to lowercase letters. Such part-of-speech tagging may be an indicator for performing emotion analysis by recognizing nouns and adjectives.

Subsequently, the control unit 250 performs labeling of text data. To this end, the control unit 250 generates a text corpus. The control unit 250 labels the corpus for each type of social network service. The control unit 250 performs keyword-based labeling based on a circumplex model of emotions. The control unit 250 classifies the labeled keyword-based text corpus based on the circumplex model of emotions. For example, the control unit 250 may classify the text corpus based on emotions such as disappointment, sadness, depression, happiness, excitement, joy, pleasure and the like. The control unit 250 stores the text corpus that is classified based on emotions in the memory 240.

The control unit 250 may apply a text corpus to a deep learning algorithm in order to obtain a context of words constituting the text corpus that is generated after labeling is completed, that is, to perform word embedding. In this case, word2vec, fast text and BERT (bidirectional encoder representation from transformers) may be applied to the deep learning algorithm used in the present invention.

When word embedding is completed, the control unit 250 performs emotion classification on each word. More specifically, the control unit 250 may apply a Bi-LSTM (bidirectional long short-term memory) neural network which is composed of LSTM units that act in both directions to combine past and future context information from text data collected from the service server 300 so as to perform emotion classification.

The control unit 250 may check the mental health status of a user who has uploaded text data related to depression and anxiety among the text data that is uploaded to the service server 300 based on the result of emotion classification performed by using the deep learning-based text analysis technique. To this end, the control unit 250 may use a circumplex model of emotions. For example, the control unit 250 may visualize words associated with an actual positive case related to depression or anxiety(depression-related content) and an actual negative case related to non-depression or non-anxiety(standard content collected by using keywords related to happiness and excitement) that are frequently identified in text data by using a word cloud. The control unit 250 may check the number of incorrect predictions and the number of correct predictions by using an error matrix. Based on this, the control unit 250 may check the mental health status of the user.

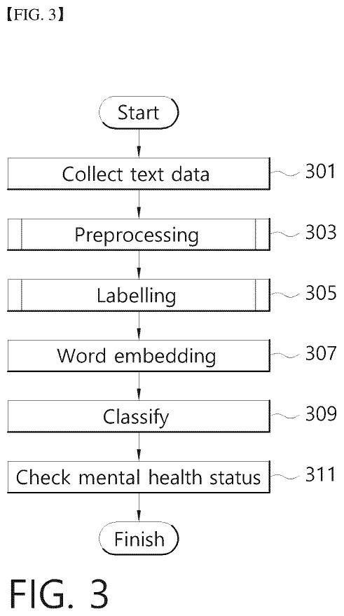

FIG. 3 is a flowchart for explaining the method for checking mental health using contents according to an exemplary embodiment of the present invention.

Referring to FIG. 3, in step 301, the control unit 250 collects text data. More specifically, the control unit 250 collects content, that is, text data from at least one service server 300 providing different social network services. In this case, the text data may refer to text data that is uploaded to the service server 300 by using at least one user terminal 100.

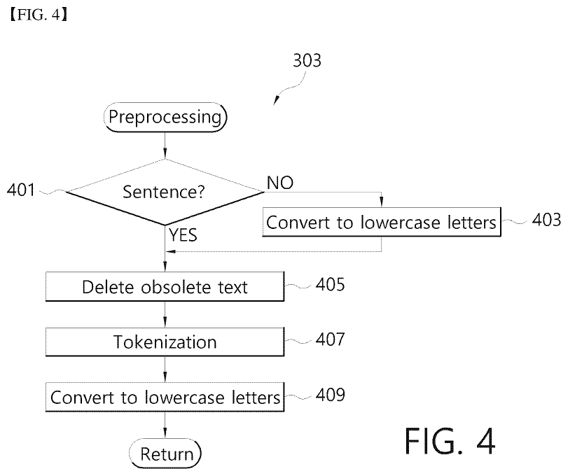

In step 303, the control unit 250 performs preprocessing on the collected text data. In this case, the method of performing preprocessing on text data will be described in more detail with reference to FIG. 4 below. FIG. 4 is a detailed flowchart for explaining the method of preprocessing data according to an exemplary embodiment of the present invention.

Referring to FIG. 4, in step 401, the control unit 250 checks whether the text data collected in step 301 is a sentence. As a result of checking in step 401, if the text data is a sentence, the control unit 250 performs step 405, and if the text data is not a sentence, for example, a paragraph, the control unit 250 performs step 403. In step 403, the control unit 250 divides the collected paragraph text data into at least one sentence and performs step 405.

In step 405, the control unit 250 analyzes text data in units of sentences and deletes obsolete text. For example, the control unit 250 deletes obsolete text such as hash tags, special characters, numbers and spaces included in the text data in order to filter and organize messy text data.

In step 407, the control unit 250 performs tokenization. More specifically, the control unit 250 removes meaningless text such as pronouns, prepositions, symbols, conjunctions, articles and URLs from text data. The control unit 250 may classify the text data from which meaningless text has been removed to check headwords or morphemes, and convert slang and emoticons included in the text data into words with the same meaning to perform tokenization.

In step 409, the control unit 250 converts all words, for example, words identified as headwords or morphemes, words converted from slang words and emoticons and the like to lowercase letters to have a certain format in order to avoid confusion that may occur during the analysis of text data, and performs step 305 of FIG. 3. Moreover, the control unit 250 may tag parts of speech such as nouns, verbs, adjectives, adverbs, determiners and conjunctions to the words converted to lowercase letters. Such part-of-speech tagging may be an indicator for performing emotion analysis by recognizing nouns and adjectives.

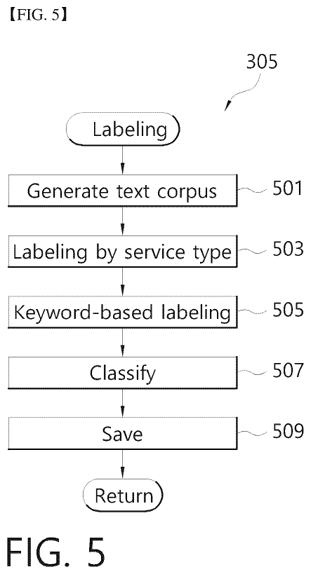

Next, in step 305, the control unit 250 performs labeling of the text data. In this case, the operation of labeling text data will be described in more detail with reference to FIG. 5 below. FIG. 5 is a detailed flowchart for explaining the method of labeling data according to an exemplary embodiment of the present invention.

Referring to FIG. 5, in step 501, the control unit 250 generates a text corpus and performs step 503. In step 503, the control unit 250 labels the text corpus by the type of social network service and performs step 505. In step 505, the control unit 250 performs keyword-based labeling based on a circumplex model of emotions. In step 507, the control unit 250 classifies the labeled keyword-based text corpus based on the circumplex model of emotions as emotion-based. For example, the control unit 250 may classify the text corpus based on emotions such as disappointment, sadness, depression, happiness, excitement, joy and pleasure. In step 509, the control unit 250 stores the text corpus classified based on emotions and performs step 307 of FIG. 3.

Subsequently, in step 307, the control unit 250 may apply the text corpus to a deep learning algorithm in order to obtain a context of words constituting the text corpus generated in FIG. 5, that is, to embed words. In this case, word2vec, fast text, and BERT (bidirectional encoder representation from transformers) may be applied to the deep learning algorithm used in the present invention.

First, when the word2vec algorithm is applied, the control unit 250 accepts a large amount of text corpus and generates vectors associated with the text corpus. In this case, the vector for each word included in the text corpus is a method of locating words having a common context close to each other in the text corpus. In the present invention, a skip-gram model may be used among representative models of the word2vec algorithm, and the skip-gram model may be trained by an improved task for consistent words.

In order to find a word representation that can infer neighboring words within context c with high accuracy, the skip-gram model improves the next objective of the average log-probability over all target words N and their contexts, for a given set of words {ω₁, θ₂, ω₃, . . . , ωₙ}. This is shown in Mathematical Formula 1 below.

$$\frac{1}{N} = {\sum }_{n = 1}^{N} {\sum }_{- v \leq j \leq {v}_{j} \neq 0} log P ( {ω}_{n + j} \vert {ω}_{n} ) \qquad [ Mathematical Formula 1 ]$$

The predicted probability for a context word may be based on the inner product of the vector representation of each of the input and output candidates {ω₁ and ω₀}. Each is normalized to conform to the requirement of a probability distribution for all words in a vocabulary of size N by using the softmax function. In this case, the softmax function is shown in Mathematical Formula 2 below.

$$P ( {ω}_{o} \vert {ω}_{i} ) = \frac{exp ( {v}_{{ω}_{0}}^{T} {v}_{{ω}_{I}} )}{{\sum }_{ω = 1}^{W} exp ( {v}_{ω}^{T} {v}_{{ω}_{I}} )} \qquad [ Mathematical Formula 2 ]$$

However, the complexity of determining these probabilities and correlation slopes for every word comes at a cost as the vocabulary grows. Therefore, in the present invention, all text data collected from the service server 300 is transferred to the neural network, and a model is trained to represent the collected text.

Second, when fast text is applied, the control unit 250 may perform word embedding by considering each word as a collection of subwords and enabling a given word to have more morphological forms. In this case, for simplification purposes and language independence, subwords are considered as n-grams of letters in words. In addition, the vector of a word is simply considered as the sum of all vectors of the word's constituent character n-grams. In this case, n-gram means a method of cutting the input word string into n syllable units.

Third, by applying BERT, the control unit 250 may use a converter structure as an attention mechanism for discovering contextual contact between words (or subwords) in text. A transformer in the basic form may include two separate components: an encoder for reading text input, and a decoder for generating predictions for a task. In this case, since the main goal of BERT is to create a language model, only an encoder mechanism is required. During training, BERT may use the methods of masked language model (MLM) and next sentence prediction (NSP) to grasp the contextual meaning of words in left and right directions.

In the case of using the MLM method, the control unit 250 masks 15% of the words of each sequence before transferring the input sequence to the BERT model. Afterwards, the control unit 250 predicts what the masked word is based on the context provided by other words in the sequence. In this case, word prediction may be performed by arranging a classification layer above the encoder output, converting into a vocabulary dimension by multiplying the output vector by an embedding matrix, and calculating the probability of each word in the vocabulary by using softmax.

In the case of using the NSP method, the control unit 250 may receive a pair of sentences as input by using the BERT model, and find several relationships that can predict whether the second sentence in the pair is the next sentence in the original document. In the training stage, half of the inputs may be the second sentence being the resulting sentence from the original document, and the other half of the inputs may be a random sentence identified from the corpus chosen as the second sentence. In this case, BERT may be an encoder stack of a transformer structure. The transformer structure is an encoder-decoder network that uses self-attention on the encoder side and attention on the decoder side.

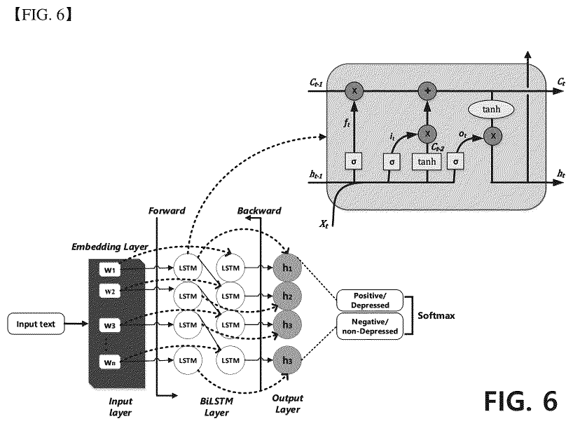

As such, when word embedding is completed by using any one of word2vec, fast text and BERT, that is, the algorithm, in step 309, the control unit 250 performs emotion classification based on machine learning or deep learning by using vectors for each word identified in step 307. The control unit 250 may apply a bidirectional long short-term memory (Bi-LSTM) neural network composed of LSTM units acting in both directions to combine past and future context information from the text data collected in step 301. Bi-LSTM may learn long-term dependencies without maintaining redundant context information. FIG. 6 is a diagram for explaining the Bi-LSTM operation according to an exemplary embodiment of the present invention.

Referring to FIG. 6, each LSTM unit may include a hidden layer that is capable of retaining previous information for a longer period of time. The main element of the LSTM unit is a memory cell Cₜ, and the memory cell may be updated by using an input gate iₜ and a deletion gate fₜ. In this case, the input data plays a role of determining what information should be stored in the memory cell, and the deletion gate plays a role of determining what information should be discarded in the memory cell. At each time step, the memory cell for the forward LSTM may be updated through Mathematical Formula 3 below.

Cₜ=fₜ*Cₜ₋₁+iₜ*Cₜ₋₂ [Mathematical Formula 3]

Moreover, fₜ is shown in Mathematical Formula 4 below, and iₜ is shown in Mathematical Formula 5 below. In addition, Cₜ₋₂ is the same as show in Mathematical Formula 6 below.

fₜ=σ(ωf*(hₜ₋₁,xₜ)+b_{f}) [Mathematical Formula 4]

iₜ=α(ωᵢ*(hₜ₋₁,xₜ)+bᵢ) [Mathematical Formula 5]

Cₜ₋₂=tahnh(ω_{c}*hₜ₋₁,xₜ)+b_{c}) [Mathematical Formula 6]

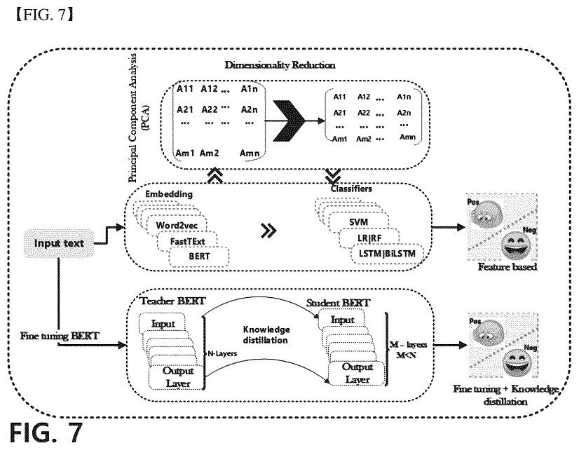

The control unit 250 performs principal component analysis (PCA) before applying the vector for each word identified due to the completion of word embedding to the classifier. In this case, PCA refers to a technique used to reduce the dimensionality of a data set that is composed of many variables related to each other while maintaining the transformation available in the data set. This will be explained by using FIG. 7. FIG. 7 is a diagram for explaining text representation, dimensionality reduction, and depression and anxiety sensing operations according to an exemplary embodiment of the present invention.

The control unit 250 may use a bi-directional implementation approach method as shown in FIG. 7 to classify posts (positive/negative) related to depression and anxiety by using a deep learning-based text analysis technique. The control unit 250 may perform a machine learning or deep learning algorithm for the purpose of detecting depression or anxiety together with dimensionality reduction to maximize accuracy. In addition, the control unit 250 may construct a lighter and smarter model by applying fine-tuning and model optimization (knowledge distillation). Knowledge distillation may compress a model by training an operation to be performed step-by-step by using an already trained network, such as the BERT model, in a smaller network.

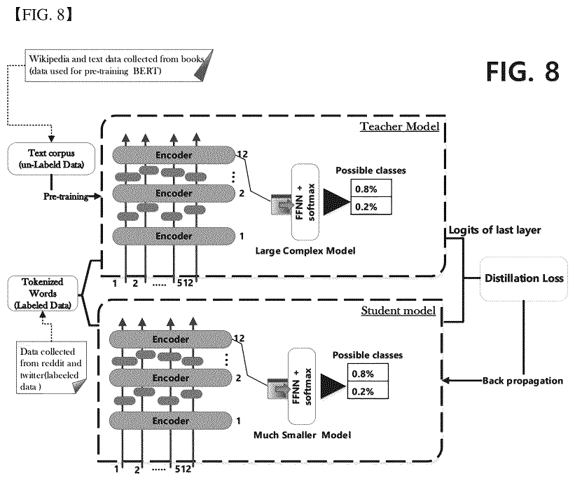

An emotion analysis model based on such knowledge distillation is shown in FIG. 8 below. FIG. 8 is a diagram showing the detailed structure for applying knowledge distillation in a pre-trained BERT according to an exemplary embodiment of the present invention.

FIG. 8 may show how weights are applied to the pre-trained BERT to build a fine-tuned and model-optimized model. In the case of the teacher-student structure used in knowledge distillation, the quality of knowledge acquisition and extraction from teachers to students depends on how the teacher-student network is designed. Guidance signals from the teacher model, commonly referred to as “knowledge” learned by the teacher model, assist the student model to simulate the behavior of the teacher model. In sentiment analysis tasks, logits, for example, the output of the last layer of a deep neural network, are used to convey knowledge from the teacher model that is not explicitly provided in the training data sample. In this case, if the logit vector z is given as the output of the last fully connected layer of the deep model, zᵢ is the logit for the i-th class, and the probability pᵢ that the input belongs to the i-th class may be estimated by Mathematical Formula 7, which is the softmax function shown below.

$${p}_{i} = \frac{ext ( {z}_{i} )}{{\sum }_{i} exp ( {z}_{i} )} \qquad [ Mathematical Formula 7 ]$$

As such, the prediction of the soft target obtained from the teacher model includes dark knowledge, and it may be utilized as a supervisor to transfer knowledge from the teacher model to the student mode. To this end, the control unit 250 may perform the operations of teacher network training, communication setting, delivery through the teacher network and backpropagation through the student network.

Teacher network training refers to an operation in which a highly complex teacher network is separately trained on a GPU with high computing power and high performance. Communication setting refers to an operation of directly delivering the output of a layer in the teacher network to the student network or performing some data augmentation before delivering to the student network. Delivering through the teacher network means an operation that can be applied when there is data augmentation to the same result after obtaining all intermediate outputs by delivering data through the teacher network. Backpropagation through the student network means an operation in which the student network learns how to replicate the operation of the teacher network by using a correspondence relationship between the output of the teacher network and the error backpropagation of the student network.

The reaction-based distillation loss as described above may be expressed as shown in Mathematical Formula 8 below.

L_{D}(p(zₜ),p(zₐ))=L_{KL}(p(zₐ),p(zₜ)) [Mathematical Formula 8]

In this case, L_{KL} denotes the Kullback-Leibler divergence loss. The Kullback-Leibler divergence score or KL divergence score quantifies how much one probability distribution differs from another probability distribution. In the present invention, the KL divergence between two distributions Q (teacher model) and P (student model) is denoted as KL(P∥Q), and the II operator may represent divergence or Ps divergence from Q. The KL divergence may be calculated by multiplying the negative sum of the probabilities of each event in P by the logarithm of the probability of an event in Q over the probability of an event in P. This is shown in Mathematical Formula 9 below.

KL(P∥Q)=−sum x in XP(x)*log(Q(x)/P(x) [Mathematical Formula 9]

Based on this loss calculation, the control unit 250 may check how the student network replicates the operation of the teacher network by backpropagation.

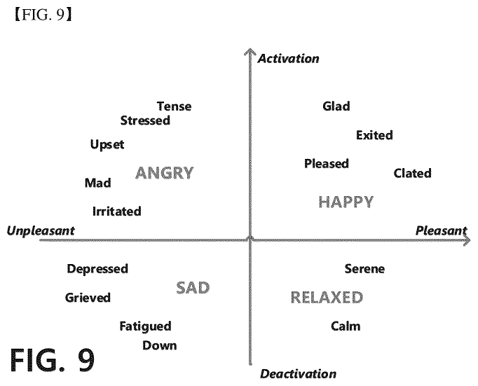

In step 311, the control unit 250 may check the mental health status of a user who has uploaded the content to the service server 300 based on posts (positive/negative) related to depression and anxiety by using a deep learning-based text analysis technique. To this end, the control unit 250 may use a circumplex model of emotions as shown in FIG. 9. FIG. 9 is a diagram showing the circumplex model of emotions according to an exemplary embodiment of the present invention.

As shown in FIG. 9, the control unit 250 may visualize words related to an actual positive case related to depression or anxiety(depression-related content) and an actual negative case related to non-depression or non-anxiety(standard content collected by using keywords related to happiness and excitement) that are frequently identified in text data by using a word cloud. The control unit 250 may check the number of incorrect predictions and the number of correct predictions by using an error matrix. Based on the error matrix, accuracy, precision and recall may be calculated by using Mathematical Formulas 10 to 12 below.

$$accuraty = \qquad [ Mathematical Formula 10 ]$$

$$\frac{truepositive + truenegative}{truepositive + negative + falsepositive + fa lsenegative}$$

$$precision = \frac{truepositive}{truepositive + falsepositive} \qquad [ Mathematical Formula 11 ]$$

$$recall = \frac{truepositive}{truepositive + falsenegative} \qquad [ Mathematical Formula 12 ]$$

In this case, P indicates an actual positive case related to depression or anxiety according to the present invention, and N indicates an actual negative case related to non-depression or non-anxiety. In addition, true positive (TP) indicates a case where the actual class of the collected text data is true(1) and the predicted class is true(1), and true negative (NP) indicates a case where the actual class of text data is false and the predicted class is false. In addition, a false positive (FP) indicates a case where the actual class of text data is false(0) and the predicted class is true(1), and a false negative (FN) indicates a case where the actual class of the text data is true(1) and the predicted class is false(0).

The exemplary embodiments of the present invention disclosed in the present specification and drawings are only presented as specific examples to easily explain the technical content of the present invention and help understanding of the present invention, and are not intended to limit the scope of the present invention. Therefore, the scope of the present invention should be construed as including all changes or modifications derived based on the technical idea of the present invention, in addition to the exemplary embodiments disclosed herein.

1. A method for checking mental health using contents, the method comprising:
collecting text data, which is content uploaded to at least one service server providing a social network service, by an electronic apparatus;
performing preprocessing by which the electronic apparatus removes obsolete text from the text data and converts the extracted meaningful text to lowercase letters;
performing, by the electronic apparatus, labeling of the preprocessed meaningful text;
performing, by the electronic apparatus, word embedding for the labeled meaningful text; and
checking the mental health status of a user who has uploaded the text data by applying the word embedding result to a deep learning algorithm by the electronic apparatus.

2. The method of claim 1, wherein the performing preprocessing comprises:
dividing a paragraph into a plurality of sentences when the text data is a paragraph.

3. The method of claim 2, wherein the performing preprocessing comprises:
removing obsolete text including hash tags, special characters, numbers and spaces from the text data;
tokenizing by classifying at least one text included in the text data into words; and
converting the meaningful text into lowercase letters.

4. The method of claim 3, wherein the tokenizing comprises:
removing meaningless text including pronouns, prepositions, conjunctions, articles and URLs from the text data;
checking a headword or morpheme based on at least one text classified as the word; and
converting slang and emoticons included in the text data into words having the same meaning.

5. The method of claim 3, further comprising:
displaying parts of speech including nouns, adjectives, adverbs, determiners and conjunctions in the meaningful text.

6. The method of claim 5, wherein the performing labelling of the meaningful text comprises:
generating a text corpus based on the meaningful text;
labeling the text corpus for each social network service;
performing keyword-based labeling based on a circumplex model of emotions; and
classifying the text corpus according to emotion based on the labeling.

7. The method of claim 1, wherein the performing the word embedding is a performing the word embedding by applying the labeled meaningful text to a BERT algorithm, which is the deep learning algorithm.

8. An apparatus for checking mental health using contents, comprising:
a communication unit for collecting text data, which is content uploaded to a service server through communication with at least one service server providing social network service; and
a control unit for performing preprocessing to convert meaningful text extracted by removing obsolete text from the text data into lowercase letters, labelling the preprocessed meaningful text, and checking the mental health status of a user, who has uploaded the text data, by applying a word embedding result for the labeled meaningful text to a deep learning algorithm.

9. The apparatus of claim 8, wherein when the text data is a paragraph, the control unit divides the paragraph into a plurality of sentences.

10. The apparatus of claim 9, wherein the control unit removes obsolete text including hash tags, special characters, numbers and spaces from the text data, and tokenizes by classifying at least one text included in the text data into words.

11. The apparatus of claim 10, wherein the control unit removes meaningless text including pronouns, prepositions, conjunctions, articles and URLs from the text data, checks a headword or morpheme based on at least one text classified as the word, and converts slang and emoticons included in the text data into words having the same meaning to perform the tokenizing.

12. The apparatus of claim 11, wherein the control unit displays parts of speech including nouns, adjectives, adverbs, determiners and conjunctions in the meaningful text.

13. The apparatus of claim 12, wherein the control unit converts the meaningful text into lowercase letters.

14. The apparatus of claim 13, wherein the control unit generates a text corpus based on the meaningful text, performs labeling of the text corpus for each social network service, performs keyword-based labeling based on a circumplex model of emotions, and classifies the text corpus according to emotion based on the labeling.

15. The apparatus of claim 14, wherein the control unit performs the word embedding by applying the labeled meaningful text to a BERT algorithm, which is the deep learning algorithm.
